(19) United States
(12) Patent Application Publication
(10) Pub. No.: US 2022/0134122 A1
(43) Pub. Date: May 5, 2022

(54) Method of Treatment Using Nuclear Magnetic Photon Excitation

(71) Applicant: Bradley Zang, Jacksonville, FL (US)

(72) Inventor: Bradley Zang, Jacksonville, FL (US)

(21) Appl. No.: 17/089,940

(22) Filed: Nov. 5, 2020

(51) Int. Cl. A61N 2/00; A61N 2/02; A61M 1/14; A61K 41/00; A61K 33/00

(52) U.S. Cl. CPC A61N 2/004; A61N 2/02; A61M 2202/0007; A61K 41/0052; A61K 33/00; A61M 1/14

(57) ABSTRACT

This invention is a method of treatment for inhibiting the cells of an infection from performing synthesis, while allowing the immune system within the infected host to maintain cell proliferation. This is accomplished by infusing an element isotope into the blood stream of the infected host. The element isotope must be capable of attaching or binding to the site of DNA or RNA polymerase. Thus, when a magnetic field and targeted low energy electromagnetic radiation are applied to substantially the location of the infection, the cells of the infection are inhibited from replicating. Therefore, the cells of the infection are prevented from replicating, which allows the host's immune system to more efficiently destroy the infectious cells through the natural process of producing antibodies.

### RELATED APPLICATIONS

The present nonprovisional continuation in part patent application claims the benefit of the filing date under 35 U.S.C. § 119(e) of Provisional Patent Application Ser. No. 62/536,662 filed on Jul. 25, 2017 and the prior filed nonprovisional application filed on Jul. 25, 2018 with associated Ser. No. 16/045,093.

### FIELD OF THE INVENTION

This invention relates to a method of treatment for bacterial and viral infections, as well as, cancers and other fast cell proliferation using nuclear magnetic photon excitation. While many different element isotopes can be utilized within the claimed method herein, the isotope, Magnesium-25, is the best element for this method of treatment.

### PRIOR ART

There are many other prior art references that discuss treatment options to teach the use of isotopes to treat bacteria or viruses. None of the prior art references teach the combination of the use of an isotope and photon excitation to retard the replication of harmful bacteria cells.

### BRIEF SUMMARY OF THE INVENTION

When the human body becomes infected with a harmful bacteria or virus (antigen), the body will naturally produce antibodies that fight the antigens to return the body to a normal, healthy state. However, many bacteria or viruses replicate much faster than the antibodies for the host. If the rate of replication is left unchecked, the harmful bacteria or virus will eventually endanger the host.

The method of treatment described and claimed herein is intended to target an infected area within the body and slow down an infection's or cancer's growth rate, thereby allowing a patient's immune system to defend against infection or cancer progression. While this method of treatment may be used on various infections and cancers, the method of treatment will be described herein as being used to treat an infection and for this application a bacterial infection specifically. The method will also be used to treat viral infections.

Bacteria are harmful cells that attack the normal, healthy cells within the body. The body produces its own cells that fight off bacteria to maintain healthy cell growth as well as replacing cells. When an infection through a bacteria enters the body it will replicate at various rates depending on the nature of the bacteria. When the body senses the introduction of a harmful bacteria (antigen), the body will produce cells (antibodies) to fight against the harmful bacteria.

It is important to slow the rate of harmful bacteria cell replication to allow the body time to produce its own cells to fight the bacterial infection. Many bacterial infections are treated with antibiotics and this application will slow the rate of growth so that the dosage of the antibiotic may be decreased.

Known methods of treatment use high energy photon electromagnetic radiation or ionizing radiation to defend against cancerous cells within a patient. While the ionizing radiation may destroy cancerous cells, the radiation will also destroy or significantly damage healthy surrounding tissue cells of the patient.

The method described and claimed herein works to reduce cell reproduction and eventually stop cell proliferation by inhibiting DNA replication of the harmful bacteria or virus. After a period of time, the infection will cease to exist because the cells causing the infection will be unable to conduct RNA synthesis or die from immune system attacks. The claimed method of treatment includes infusing an element isotope in the blood stream and other fluids such as Cerebrospinal fluid for Meningitis infections of a patient, by substantially locating the areas or source of infection within a patient, providing a magnetic field substantially at the location of the infection, and transmitting low energy radio frequencies substantially at the location of the infection.

While the preferred isotope for the claimed method of treatment is Magnesium-25, it is anticipated that other isotopes may be utilized. In this application the Magnesium-25 is introduced either as a supplement to a person's diet either through fertilizer or added to the processed food. The introduction of the Magnesium-25 into the person can also be achieved through an intravenous drip.

Once the Magnesium-25 is introduced, photon excitation is used to enable the Magnesium-25 to attach to the harmful bacteria or virus. Once the Magnesium-25 attaches to the harmful bacteria it prevents the replication of the harmful bacteria.

### BRIEF DESCRIPTION OF THE DRAWINGS

FIG. 1 is a diagram which displays the steps of the method of treatment.

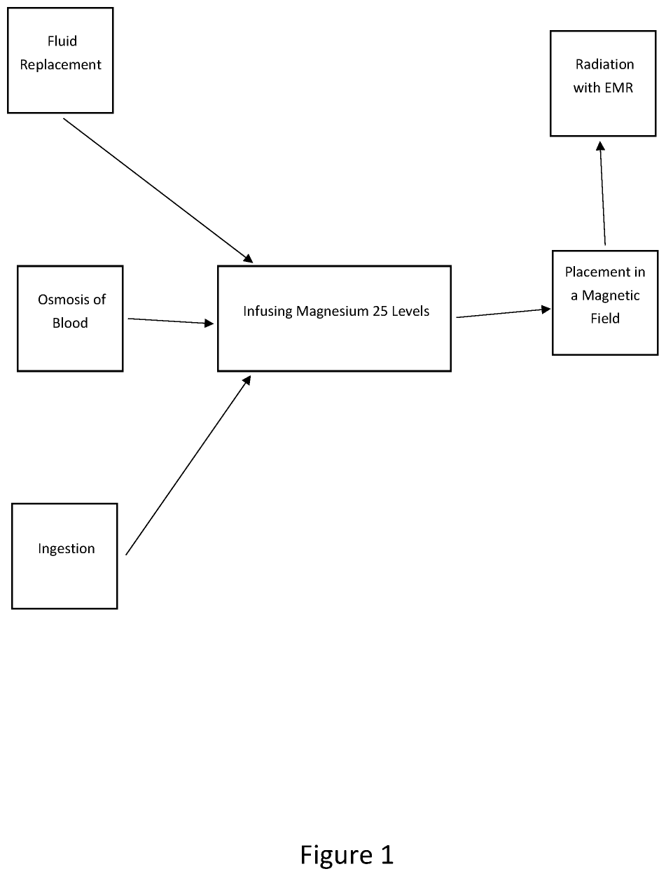

FIG. 2 is a diagram which displays a second embodiment for steps of the method of treatment.

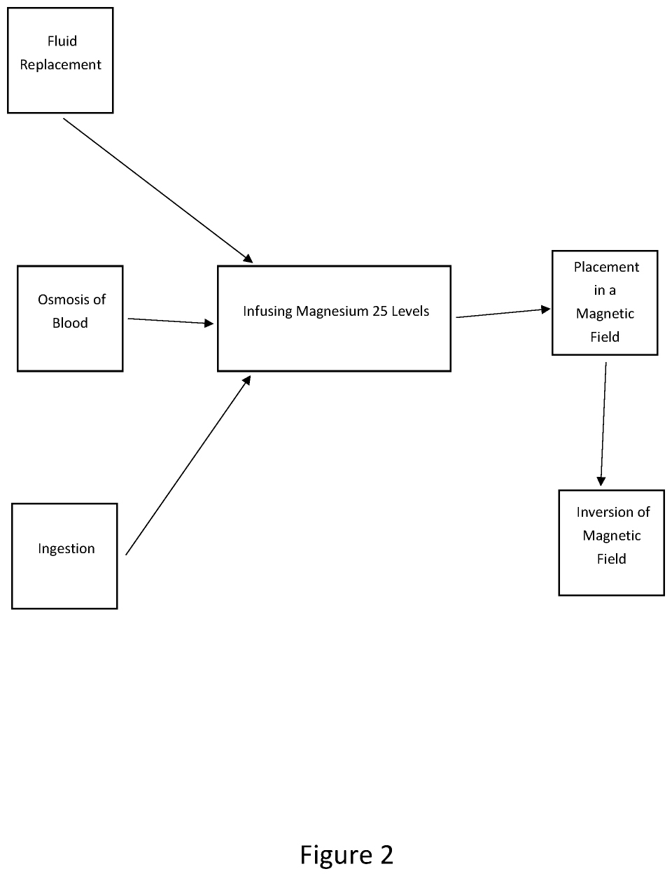

FIG. 3 is an isometric view of a host siting in a chair where a magnetic field generator is providing a magnetic field engulfing the infected area within the host and a plurality of transmitters are supplying low energy electromagnetic frequencies at substantially the location of the infection.

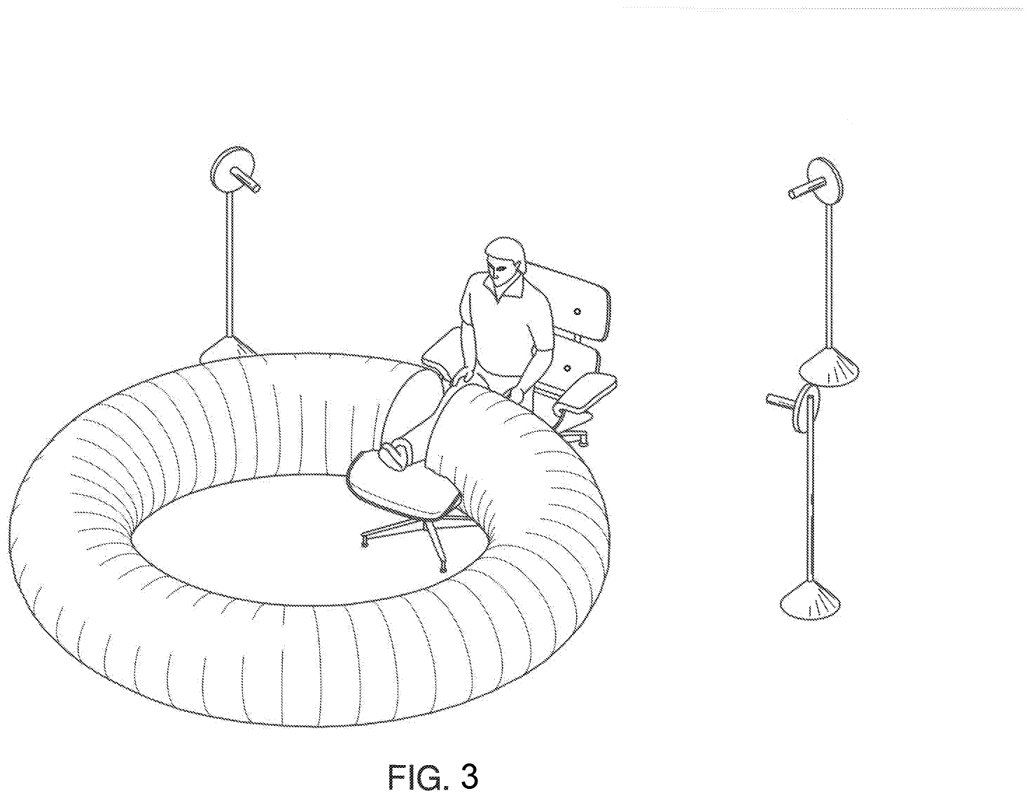

### DETAILED DESCRIPTION OF THE EMBODIMENTS

The human body constantly produces normal healthy cells. When the body is subjected to an infection or antigen (either from a bacteria or a virus) the body will begin to produce antibodies to ward off the infection. However, many bacteria and viruses replicate at higher rates than the antibodies that are produced by the body naturally. If the rate of replication of bacteria or virus is left unchecked, it will endanger the entire body.

This invention is a method of treatment for reducing the growth rate and progression of an infection or cancer to thereby stop cell proliferation of a harmful bacteria or virus and allow a patient's immune system to effectively defend and protect the patient or host. This method of treatment is a unique and novel procedure of poisoning a catalyst effect by replacing it with its magnetic isotope. The method of treatment begins with raising the magnetic isotope level of a patient's blood through ingestion of a nonradioactive isotope element, dialysis, or blood osmosis of a nonradioactive isotope element. The preferred nonradioactive isotope element for the method of treatment claimed and described herein is Magnesium-25. However, other element isotopes, such as but not limited to, Nitrogen-15, can be used with this method of treatment.

The infused Magnesium-25 is then absorbed by the cells of the infection within the patient and bound to the DNA polymerase and RNA polymerase of the cells.

Once the Magnesium-25 is infused into the patient's blood and absorbed by the cells of the infection, the location of infection should be identified or the substantial location of infection should be identified. It is anticipated that substantially locating the infection can be performed prior to infusing Magnesium-25 into the patient's bloodstream. For people with immunity disorders, they may be placed on a Magnesium 25 diet so when an infection does occur they can immediately be treated with the fields when the infection is located. Magnesium-25 may also become part of a person's diet through the introduction of Magnesium-25 through a person's diet directly or through fertilizers that are used to grow the food that is eaten.

A magnetic field is provided around the infected area of the patient. Once the magnetic field is provided, a low frequency electromagnetic radiation is transmitted substantially at the location of the infection. For example, the magnetic field may have a 5.373 to 5.5077 tesla field. By providing the magnetic field, the cells which have absorbed the infused Magnesium-25 isotope are susceptible to accepting additional photons.

A low frequency electromagnetic radiation is transmitted substantially at the location of the infection. It is critical that low energy electromagnetic radiation, also known as low energy photons are used so other chemicals and cells, within the body do not absorb the energy. The low energy electromagnetic frequency should be between 14.000 MHz and 14.350 MHz. Nuclear magnetic frequencies is ideal for use with this method because it has too weak of a photon to affect other chemicals within the patient. However, it does affect the cell synthesis that the Magnesium-25, or other predetermined element isotope, catalyzes, so healthy slow growing cells within the body will not be destroyed.

The additional energy provided by the low energy electromagnetic radiation then excites the Magnesium-25, thereby poisoning the catalytic effect of the Magnesium-25. Because Magnesium-25 binds at the DNA polymerase and RNA polymerase sites, after it is affected by the low energy electromagnetic radiation it inhibits cell synthesis from occurring in each cell of the infection. In other words, because the Magnesium-25 ion is at a higher energy level, with photon excitation it does not allow the catalysis to effectively operate and prevents rapid replication of the harmful bacteria cells. Accordingly, the synthesis phase of the infection's cells are inhibited, which prevents infection cell proliferation.

Since only portions of the body would be exposed to the correct magnetic field and electromagnetic radiation, only those portions of the body would experience lower cell proliferation. Thus, a patient's immune system response created in other parts of the body can perform cell reproduction and travel to the location of the infection to destroy the infectious cells.

While the embodiments of the invention have been disclosed, certain modifications may be made by those skilled in the art to modify the invention without departing from the spirit of the invention.

The inventor claims:

1. A method of treatment using nuclear magnetic photon excitation which is comprised of the following steps:
a. identifying the presence of an infection in a host;
b. identifying a substantial location of an infection within the host;
c. infusing an element isotope into the host's bloodstream other fluids and cells;
d. providing a magnetic field throughout the location of the infection;
e. transmitting low energy electromagnetic frequency substantially at the location of the infection.

2. The method of treatment as described in claim 1 wherein the element isotope is Magnesium-25.

3. The method of treatment as described in claim 1 wherein the element isotope is Nitrogen-15.

4. The method of treatment as described in claim 1 wherein the element isotope is infused through dialysis.

5. The method of treatment as described in claim 1 wherein the element isotope is infused by ingestion by the host.

6. The method of claim 1 wherein the magnetic field can have a strength ranging from 5.373 tesla to 5.5077 tesla.

7. The method of claim 1 wherein the low energy electromagnetic frequency is a radio frequency.

8. The method of claim 1 wherein the low energy electromagnetic frequency ranges from 14.000 MHz to 14.350 MHz.

9. The method of claim 1 wherein the low energy electromagnetic frequency is near that of the nuclear magnetic resonance frequency for other magnetic fields for the isotope elements.

10. The method of claim 1 wherein the magnetic field is correlated with the electromagnetic frequency.

11. A method of treatment using nuclear magnetic photon excitation, which is comprising of the following steps:
a. Identifying a substantial location of an infection within a host;
b. infusing an element isotope into the host's bloodstream other fluids and cells;
c. providing a magnetic field throughout the location of the infection;
d. inverting the magnetic field.

12. The method of treatment as described in claim 11 wherein the element isotope is Magnesium-25.

13. The method of treatment as described in claim 11 wherein the element isotope is Nitrogen-15.

14. The method of treatment as described in claim 11 wherein the element isotope is infused through dialysis.

15. The method of treatment as described in claim 11 wherein the element isotope is infused by ingestion of the host.

16. The method of claim 10 wherein the magnetic field can have a strength ranging from 5.373 tesla to 5.5077 tesla.
